(19) United States
(12) Patent Application Publication
(10) Pub. No.: US 2024/0268486 A1
(43) Pub. Date: Aug. 15, 2024

(54) INSOLE COMBINED SOCK

(71) Applicant: Savannah Cantu, San Benito, TX (US)

(72) Inventor: Savannah Cantu, San Benito, TX (US)

(21) Appl. No.: 18/438,846

(22) Filed: Feb. 12, 2024

Related U.S. Application Data
(60) Provisional application No. 63/445,455, filed on Feb. 14, 2023.

(51) Int. Cl. A41B 11/00; A43B 17/14; A61F 5/34; B33Y 80/00

(52) U.S. Cl. CPC A41B 11/00; A43B 17/14; A61F 5/34; B33Y 80/00

(57) ABSTRACT

A novel insole-integrated sock is disclosed. The sock features an orthopedic, shock-absorbent insole made of foam or gel which is permanently or temporarily attached inside the sock to provide support to the foot of a user while wearing any shoe. The sock can be used with any shoe and comes in different sizes, shapes, and designs. The insole includes segmented density variations to optimize comfort and support. In some embodiments, the sock includes a smart sensor layer, including pressure sensor, temperature sensor, and accelerometer, and the layer is embedded within the insole. The insole does not slide or dislodge while in use, thereby providing a stable support to the foot of a user. The insole-integrated sock combines the convenience of socks with the comfort and support of insoles and alleviates discomfort and enhances the overall wearing experience of various types of shoes.

### CROSS-REFERENCE TO RELATED APPLICATION

The present application claims priority to, and the benefit of, U.S. Provisional Application No. 63/445,455, which was filed on Feb. 14, 2023, and is incorporated herein by reference in its entirety.

### FIELD OF THE INVENTION

The present invention generally relates to the field of shoe insoles. More specifically, the present invention relates to a novel sock with an integrated insole designed to be worn with any shoe to relieve discomfort. The insole may be made from gel or foam and can be permanently integrated or temporarily inserted into a regular sock. The insole does not slip around when used and enables users to achieve comfort without the need to purchase a new pair of shoes. Accordingly, the present disclosure makes specific reference thereto. Nonetheless, it is to be appreciated that aspects of the present invention are also equally applicable to other like applications, devices, and methods of manufacture.

### BACKGROUND

By way of background, foot discomfort is a common problem for individuals and can be caused by different factors including but not limited to plantar fasciitis, diabetes, and other medical conditions. Specifically, plantar fasciitis is a condition that causes inflammation of the plantar fascia, a band of tissue that runs along the bottom of the foot. Diabetes can damage nerves and blood vessels in the feet, which can lead to pain, numbness, and tingling. Further, foot pain can limit an individual's ability to walk for extended periods. Walking can be painful and uncomfortable for them, restricting their mobility and making it difficult to engage in activities that involve prolonged standing or walking.

Due to foot discomfort, individuals find it challenging to wear certain types of footwear. Different footwear such as fashionable footwear are generally uncomfortable and may cause pain to an individual with foot discomfort. Individuals often have to settle for practical and comfortable shoes that may not be very stylish or visually appealing. Oftentimes simple, and even orthopedic footwear are not comfortable for individuals with foot discomfort. Some individuals with foot discomfort alleviate their pain by using insoles or orthotic inserts. However, such inserts shift and dislodge while being used inside shoes and other footwear. This leads to discomfort or inconvenience as intended support is not provided. Purchasing multiple pairs of shoes is not only expensive but also time consuming. Conventional socks do not provide any additional support to the foot of a user while walking and individuals desire improved socks that have supporting insoles to increase functionality of the socks. Individuals desire an improved device that allows users to wear shoes that may have otherwise been uncomfortable to wear.

Therefore, there exists a long felt need in the art for a sock with an insole. Additionally, there is a long felt need in the art for an improved sock that has a permanently or temporarily attached ergonomic insole thereto. Moreover, there is a long felt need in the art for a sock with integrated insole that provides support and comfort to a user while walking, running, and doing physical activities. Further, there is a long felt need in the art for an improved sock that has an insole which can be used with any type of shoes. Furthermore, there is a long felt need in the art for an insole combined sock that provides a stable insole which does not slide or dislodge while walking and running. Finally, there is a long felt need in the art for an advanced and multifunctional footwear solution that combines the convenience of socks with the comfort and support of insoles.

The subject matter disclosed and claimed herein, in one embodiment thereof, comprises an insole-integrated sock designed for being worn with any footwear. The sock includes a cuff portion for user wearability, a foot portion with a flat seam toe section, and an orthopedic and soft insole positioned within a foot holding space of the sock. The insole is adhered to the inner bottom surface of the foot portion. The insole is constructed from a soft and comfortable material, selected from a group consisting of gel and foam, for providing shock absorption during walking, running and other physical activities. The insole has anti-microbial properties and can be integrated or detachably attached to the inner bottom surface of the sock. The insole can be adhered using a temporary or permanent adhesive and can be sewn to the sock. The insole-integrated sock combines the convenience of socks with the comfort and support of insoles. The sock alleviates discomfort and enhances the overall wearing experience of various types of shoes.

In this manner, the insole-integrated sock of the present invention accomplishes all of the forgoing objectives and provides users a sock with an insole. The insole is adhered to the inner bottom surface of the sock and stays in place inside of the sock. The insole provides comfort when worn with any shoe and enables users to achieve comfort without the need to purchase a new pair of shoes. The insole is made of gel, foam, or any other similar material and benefits any individual with foot discomfort for walking, running, and doing physical activities.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an insole-integrated sock designed for being worn with any footwear. The sock further comprising a cuff portion for user wearability, a foot portion with a flat seam toe section, and an orthopedic and soft insole positioned within a foot holding space of the sock. The insole is adhered to the inner bottom surface of the foot portion. The insole is constructed from a soft and comfortable material, selected from a group consisting of gel and foam, for providing shock absorption during walking, running and other physical activities. The insole has anti-microbial properties and can be integrated or detachably attached to the inner bottom surface of the sock.

In yet another embodiment, an insole designed to be permanently or temporarily placed inside a sock is disclosed. The insole when combined with the sock allows a user to wear any type of footwear without any foot discomfort. The insole includes a heel segment, a mid-foot segment, and a forefoot segment. The heel segment and the forefoot segment have a Shore Durometer in the range from about 30 to about 40, and the mid-foot segment (304) has a Shore Durometer in the range from about 20 to about 30. The insole is antimicrobial and is made of a material selected from the group consisting of foam and gel, wherein the material may have high or low density or a combination thereof. The insole is adhered permanently or temporarily inside the sock allowing a user to place foot on the insole while wearing the sock.

In a further embodiment, an intelligent insole for use in an insole-integrated sock is disclosed. The insole is permanently or detachably attached to inner bottom surface of the sock and includes a smart sensor layer embedded within the insole, the smart layer comprising a pressure sensor configured to detect pressure exerted on different parts of a user's foot while wearing the insole-integrated sock. A temperature sensor configured to detect the temperature of the user's foot skin and to provide information related to sweat and inflammation. An accelerometer configured to capture gait information and walking activity data. A mobile application is operatively connected to the insole via a wireless communication channel and the mobile application displays detected pressure information from the pressure sensors, recommend specific insole materials based on the detected pressure data and present gait information and walking activity data captured by the accelerometer.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1A illustrates a perspective view of a sock of the present invention with integrated insole showing the insole in accordance with the disclosed structure;

FIG. 1B illustrates an outer perspective view of the sock of the present invention with integrated insole in accordance with the disclosed structure;

FIG. 2 illustrates a side perspective view of the insole integrated sock of the present invention being worn by a user in accordance with the disclosed architecture;

FIG. 3 illustrates a perspective view of the insole used in the insole combined sock of the present invention in accordance with the disclosed architecture;

FIG. 4 illustrates another embodiment of the insole integrated to the insole integrated sock of the present invention in accordance with the disclosed structure; and

FIG. 5 illustrates a flow chart depicting a process of making the insole combined sock of the present invention in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a sock with an insole. Additionally, there is a long felt need in the art for an improved sock that has permanently or temporarily attached ergonomic insole thereto. Moreover, there is a long felt need in the art for a sock with integrated insole that provides support and comfort to a user while walking, running, and doing physical activities. Further, there is a long felt need in the art for an improved sock that has an insole and can be used with any type of shoe. Furthermore, there is a long felt need in the art for an insole combined sock that provides a stable insole which does not slide or dislodge while walking and running. Finally, there is a long felt need in the art for an advanced and multifunctional footwear solution that combines the convenience of socks with the comfort and support of insoles.

The present invention, in one exemplary embodiment, is an intelligent insole for use in an insole-integrated sock. The insole includes a smart sensor layer embedded within the insole, the smart layer comprising a pressure sensor configured to detect pressure exerted on different parts of a user's foot while wearing the insole-integrated sock. A temperature sensor configured to detect the temperature of the user's foot skin and provide information related to sweat and inflammation. An accelerometer configured to capture gait information and walking activity data. A mobile application is operatively connected to the insole via a wireless communication channel and the mobile application displays detected pressure information from the pressure sensors, recommend specific insole materials based on the detected pressure data and present gait information and walking activity data captured by the accelerometer.

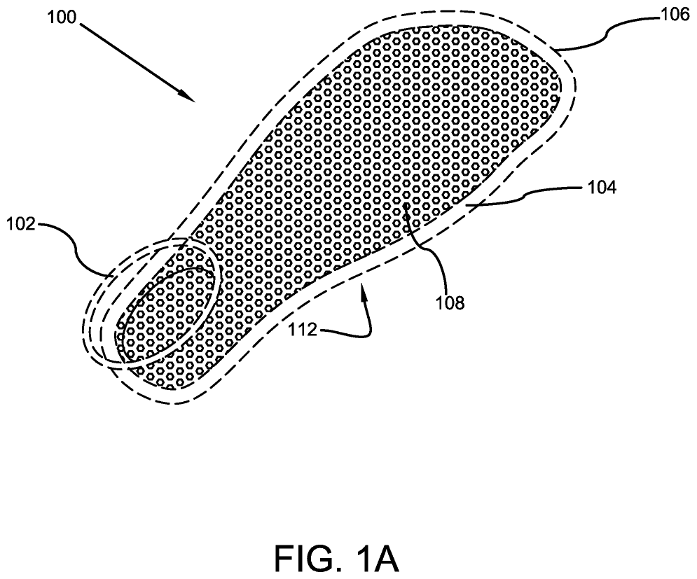

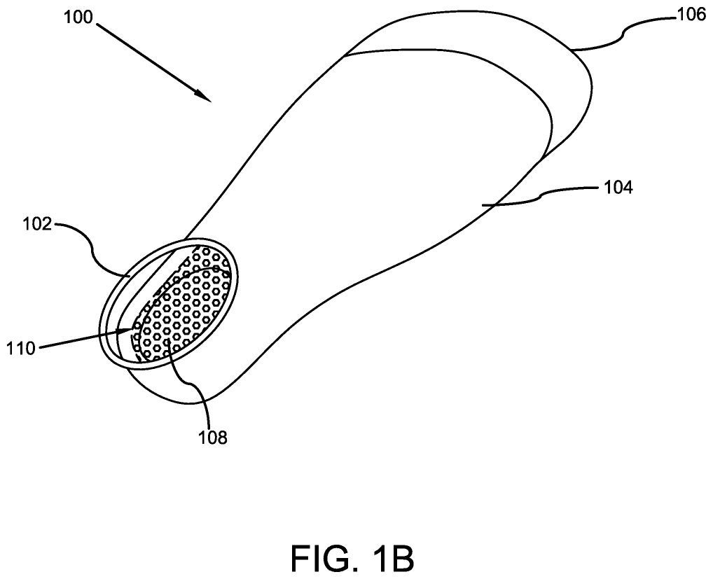

Referring initially to the drawings, FIGS. 1A and 1B illustrate perspective view of sock of the present invention with integrated insole in accordance with the disclosed structure. The insole integrated sock 100 of the present invention is designed to be worn by a user like a conventional sock while providing comfort when worn with a shoe or any other footwear. More specifically, the sock 100 in the present embodiment includes a cuff portion 102 and a foot portion 104 with a flat seam toe section 106. The cuff portion 102 can be elastic and allows a user to wear the sock 100. An orthopedic and soft insole 108 is placed inside the foot holding space 110 of the sock 100 with the insole 108 adhered to the inner bottom surface 112 of the foot portion 104. The insole 108 is made of a soft and comfortable material such as gel or foam and provides shock absorption while walking and running.

In one embodiment, the insole 108 is integrated into the sock 100 during manufacturing of the sock 100. The insole 108 can be 3D printed and integrated using a permanent adhesive. In some embodiments, the insole 108 can be sewn to the inner surface 112 of the foot portion 104. The insole 108 is machine washable and can be reused. In some embodiments, the insole 108 can be detachably attached to the sock 100, allowing a user to use the sock 100 with or without the insole 108. In different embodiments of the present invention, the insole 108 can have Shore Durometer in the range from about 20 to about 40 to meet requirements of different users.

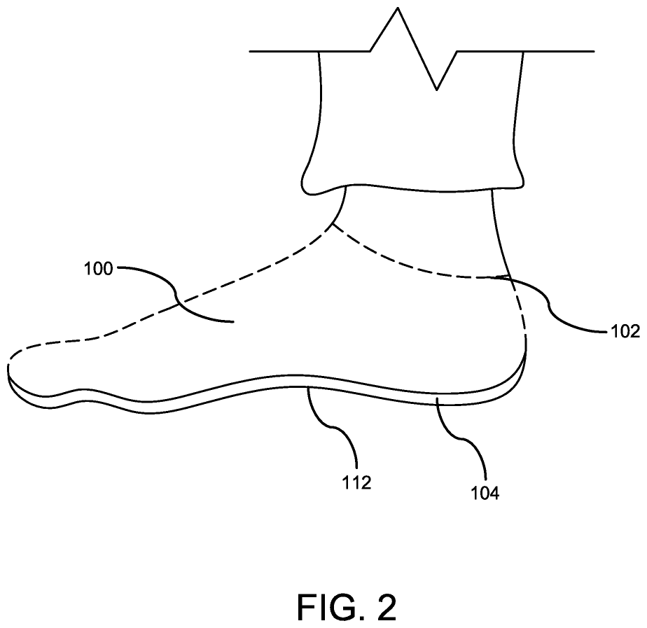

FIG. 2 illustrates a side perspective view of the insole integrated sock of the present invention being worn by a user in accordance with the disclosed architecture. The sock 100 can be worn by a user like a conventional sock by inserting a foot through the cuff 102. The foot rests on the insole 108 which is placed on the inner bottom surface 112 of the sock 100. An advantage of the sock 100 is that it can be worn with any shoes that have any type of insole. Thus, the sock 100 eliminates the requirement of purchasing different pairs of shoes having the desired insole.

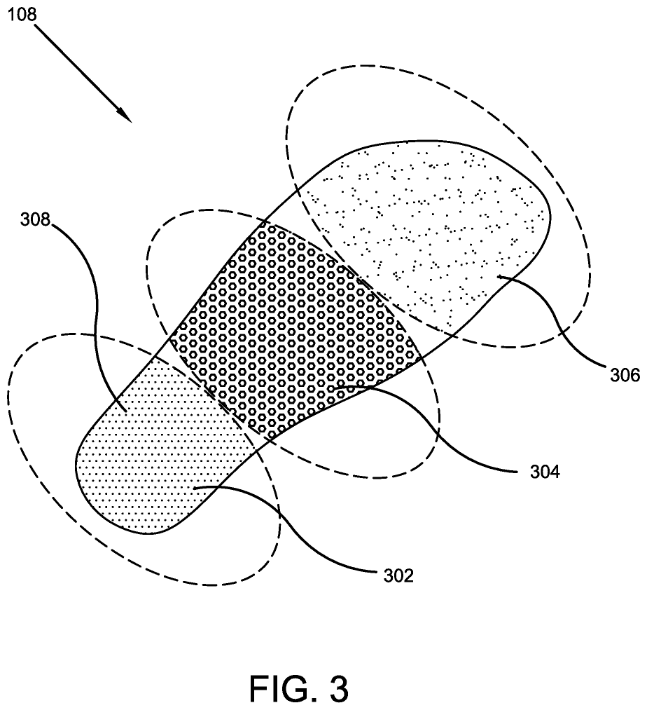

FIG. 3 illustrates a perspective view of the insole 108 used in the insole combined sock 100 of the present invention in accordance with the disclosed architecture. The insole 108 is made of foam or gel which can be of high or low density or a combination thereof. The insole 108 has a heel segment 302, a mid-foot segment 304, and a forefoot segment 306. The heel segment 302 and the forefoot segment 306 have Shore Durometer in the range from about 30 to about 40 and the mid-foot segment 304 has Shore Durometer in the range from about 20 to about 30. Further, the density of pillows or foam 308 in the segments 302, 306 is about 125% of the density of pillows or foam in the mid-foot segment 304. The insole 108 is antimicrobial and the material is odor-fighting to maintain foot hygiene.

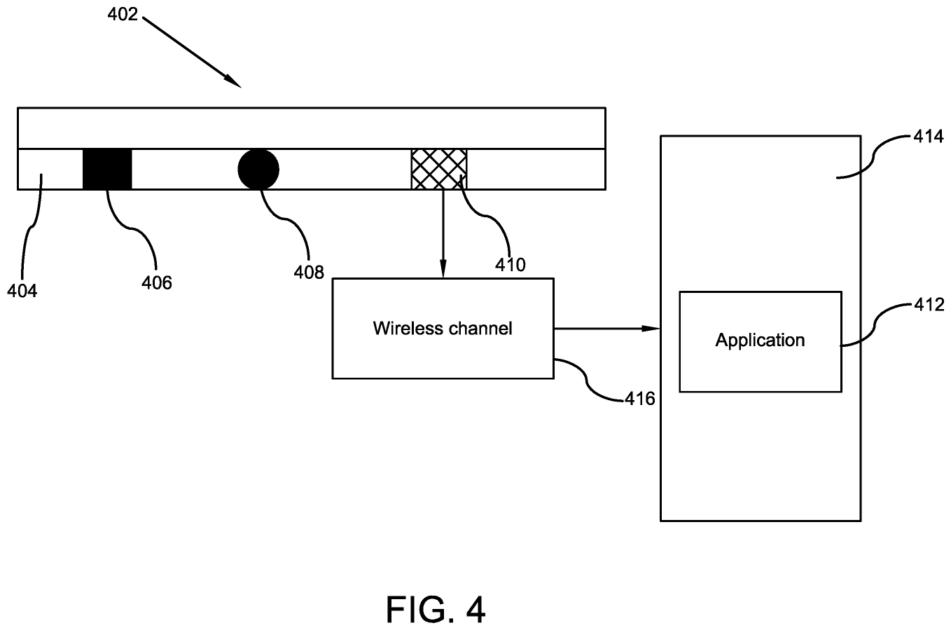

FIG. 4 illustrates a cross sectional view of another embodiment of the insole integrated to the sock of the present invention in accordance with the disclosed structure. In the present embodiment, the insole 402 includes a smart sensor layer 404 embedded within the insole 402.

The smart sensor layer 404 includes a network of pressure sensors 406, a temperature sensor 408, and an accelerometer 410. The pressure sensors 406 detect pressure exerted on different parts of foot of a user wearing the insole sock 100 allowing a user to view the detected pressure on a mobile application 412. The mobile application 412 may recommend any specific material for insole 402 based on the detected pressure. The temperature sensor 408 detects the temperature of the foot of the user to provide information about the sweat and inflammation in the skin of the foot. The accelerometer 410 provides the gait information and walking activity information which are displayed on the mobile application 412 installed in a handheld user device 414. The application 412 is connected to the insole 402 via a wireless communication channel 416 such as Bluetooth, RFID, NFC, and the like.

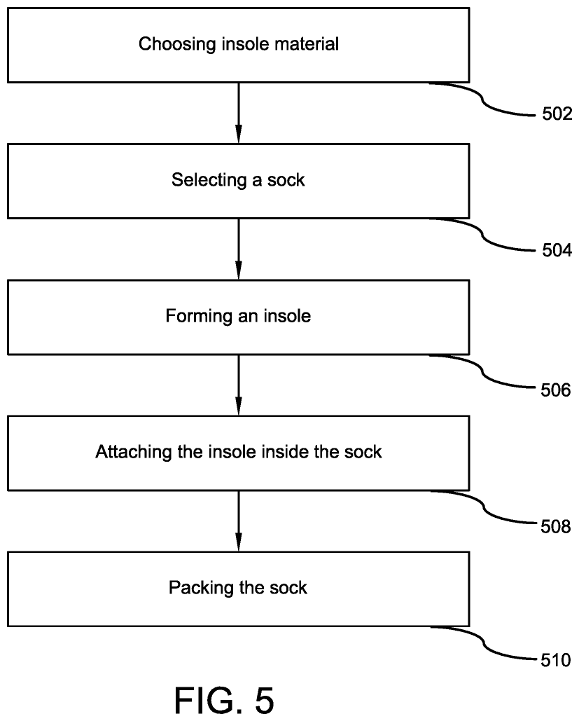

FIG. 5 illustrates a flow chart depicting a process of making the insole combined sock of the present invention in accordance with the disclosed structure. Initially, a durable, soft, and anti-microbial material is chosen to manufacture the insole of the sock (Step 502). A sock which can be of different design, shape, and size is selected for integrating or detachably attaching the insole (Step 504). The sock can be a liner sock, no show sock, ankle sock, mid-calf sock, and any other type of sock. Thereafter, the selected material is cut into the desired shape and size to form an insole corresponding to the selected sock (Steo 506). The insole can be shaped using 3D printing or any conventional tools used in the footwear industry.

Thereafter, the insole is attached to the footbed or base of the sock such that the insole is sandwiched between the foot of a user wearing the sock and the base of the sock (Step 508). The insole can be attached using an adhesive, can be sewn or can be 3D printed inside the sock. Finally, the sock is packed in packaging which may have directions of use, images of the sock, and other details printed thereon (Step 510).

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “insole integrated sock”, “insole combined sock”, “insole sock”, and “sock” are interchangeable and refer to the insole combined sock 100 of the present invention.

Notwithstanding the forgoing, the insole combined sock 100 of the present invention can be of any suitable size and configuration as is known in the art without affecting the overall concept of the invention, provided that it accomplishes the above stated objectives. One of ordinary skill in the art will appreciate that the insole combined sock 100 as shown in the FIGS. 1-4 are for illustrative purposes only, and that many other sizes and shapes of the insole combined sock 100 are well within the scope of the present disclosure. Although the dimensions of the insole combined sock 100 are important design parameters for user convenience, the insole combined sock 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An insole integrated sock comprising:
a sock having a cuff portion and a foot portion including a flat seam toe section;
wherein said cuff portion including elastic material for securing around a wearer's foot;
wherein said foot portion having a foot holding space and an orthopedic insole contained therein;
wherein said orthopedic insole is adhered to an inner bottom surface of said foot portion; and
further wherein said orthopedic insole including a material selected from the group consisting of a foam and a gel for providing shock absorption.

2. The insole integrated sock of claim 1, wherein said orthopedic insole sewn to said inner bottom surface of said portion.

3. The insole integrated sock of claim 2, wherein said sock is machine washable.

4. The insole integrated sock of claim 3, wherein said orthopedic insole detachably attached to said inner bottom surface of said foot portion.

5. The insole integrated sock of claim 4, wherein said orthopedic insole adhesively adhered to said inner bottom surface of said foot portion.

6. The insole integrated sock of claim 5, wherein said orthopedic insole is 3D printed.

7. The insole integrated sock of claim 6, wherein said orthopedic insole having a Shore Durometer from 20 to 40.

8. An insole integrated sock comprising:
a sock having a cuff portion and a foot portion;
wherein said cuff portion including elastic material for securing around a wearer's foot;
wherein said foot portion having a foot holding space and an orthopedic insole contained therein;
wherein said orthopedic insole is adhered to an inner bottom surface of said foot portion;
wherein said orthopedic insole having a heel segment, a mid-foot segment, and a forefoot segment;
wherein said heel segment and said forefoot segment having a Shore Durometer in the range from 30 to 40, and the mid-foot segment having a Shore Durometer in the range from 20 to 30; and
further wherein said heel segment and said forefoot segment having a first foam including a first foam density and said mid-foot segment having a second foam including a second foam density.

9. The insole integrated sock of claim 8, wherein said first foam density is at least 125% of said second foam density.

10. The insole integrated sock of claim 8, wherein said orthopedic insole including an antimicrobial material.

11. The insole integrated sock of claim 8, wherein said orthopedic insole sewn to said inner bottom surface of said portion.

12. The insole integrated sock of claim 8, wherein said sock is machine washable.

13. The insole integrated sock of claim 8, wherein said orthopedic insole detachably attached to said inner bottom surface of said foot portion.

14. The insole integrated sock of claim 8, wherein said orthopedic insole adhesively adhered to said inner bottom surface of said foot portion.

15. The insole integrated sock of claim 8, wherein said orthopedic insole is 3D printed.

16. The insole integrated sock of claim 8, wherein said sock is selected from the group consisting of a liner sock, a no-show sock, an ankle sock, and a mid-calf sock.

17. An insole integrated sock comprising:
a sock having a cuff portion and a foot portion;
wherein said cuff portion including elastic material for securing around a wearer's foot;
wherein said foot portion having a foot holding space and an orthopedic insole contained therein;
wherein said orthopedic insole is adhered to an inner bottom surface of said foot portion;
wherein said orthopedic insole having a heel segment, a mid-foot segment, and a forefoot segment;
wherein said heel segment and said forefoot segment having a first foam including a first foam density and said mid-foot segment having a second foam including a second foam density;
wherein said orthopedic insole having a smart sensor layer embedded within said orthopedic insole;
wherein said smart sensor layer having a plurality of pressure sensors, a temperature sensor, and an accelerometer; and
further wherein said plurality of pressure sensors detect pressure exerted on different parts of a foot of a user wearing said sock.

18. The insole integrated sock of claim 17, wherein said heel segment and said forefoot segment having a Shore Durometer in the range from 30 to 40, and the mid-foot segment having a Shore Durometer in the range from 20 to 30.

19. The insole integrated sock of claim 17, wherein said temperature sensor detecting a temperature of the foot of the user.

20. The insole integrated sock of claim 17, wherein said accelerometer providing gait information and walking activity information.
